(19) United States
(12) Patent Application Publication
(10) Pub. No.: US 2005/0276422 A1
(43) Pub. Date: Dec. 15, 2005

(54) Integral active noise cancellation section

(76) Inventors: Thomas N. Buswell, Clay, NY (US); Mark R. Rabbia, Brewerton, NY (US)

Correspondence Address: BACHMAN & LAPOINTE, P.C., 900 CHAPEL STREET, SUITE 1201, NEW HAVEN, CT, 06510, US

(21) Appl. No.: 10/852,972

(22) Filed: May 25, 2004

(51) Int. Cl. A61F 11/06; G10K 11/16; H03B 29/00

(52) U.S. Cl. 381/71.14; 381/71.1

(57) ABSTRACT

Active noise cancellation apparatus comprising a plenum through which a sound travels, and a noise cancellation unit located within the plenum the noise cancellation unit comprising an apparatus for receiving the sound, an apparatus for receiving a cancelled sound signal, an apparatus for converting the received sound and the cancelled sound signal into an output signal, and an apparatus for emitting the output signal as an output acoustic signal to cancel the sound.

### BACKGROUND OF THE INVENTION

(1) Field of the Invention

The invention relates to an apparatus, and a method for using such an apparatus, for actively canceling acoustic energy in air handler units.

(2) Description of the Related Art

Air handler units (AHU), in particular air conditioning systems, typically consist of enclosed spaces through which air flows as it is directed through a structure. The flowing air is typically modified to a desired temperature and is delivered to a location, or locations, throughout the structure where such air is desired. Such airflow is generally generated by a fan powered by a motor located within the AHU. In addition to generating airflow, such fans typically generate acoustic energy, or noise, in the range of approximately 60 Hz to 8,000 Hz. The same conduits through which the modified air flows additionally enables the unwanted propagation of such noise throughout the system terminating at a point or points where the modified air is deposited.

In order to lessen the effects of the unwanted noise generated by the fan and propagated throughout the AHU, there is often installed throughout the ducts, or plenums, of the system perforated plates and sound insulation. The perforated plates and sound insulation work in concert to dampen and absorb the acoustic energy generated by the fan, motor, and other AHU components. Unfortunately, the inclusion of insulation within the ducts or plenums of the system cause unavoidable pressure drops as the air moves throughout the system. Such pressure drops lessen the efficiency of the system. In addition, insulation is subject to degradation over time and requires considerable installation effort and maintenance. Lastly, because insulation possesses static physical characteristics, and cannot actively adapt to changes in the environment in which it is installed, any change to an AHU which results in the emission of additional or not previously experienced acoustic energy, requires potentially costly alteration of the installed insulation.

What is therefore needed is an apparatus, and method for using such an apparatus, for actively and adaptively canceling acoustic energy generated by the components of an AHU. Ideally, such an apparatus would be easy to install, require little maintenance, and would actively adapt to changes in the acoustic environment within the ducts and plenums of an AHU.

### SUMMARY OF THE INVENTION

Accordingly, it is an object of the present invention to provide an apparatus, and a method for using such an apparatus, for actively canceling acoustic energy in air handler units.

In accordance with the present invention, an active noise cancellation apparatus comprises a plenum through which a sound travels, and a noise cancellation unit located within the plenum the noise cancellation unit comprising an apparatus for receiving the sound, an apparatus for receiving a cancelled sound signal, an apparatus for emitting an acoustic output signal, and a controller adapted to convert the received sound and the cancelled sound signal into an output signal to be emitted as an output acoustic signal to cancel the sound.

In further accordance with the present invention, a method for canceling sound in an air handler unit comprises receiving the sound, emitting an output acoustic signal to cancel the sound, receiving a cancelled sound signal, and converting the received sound and the cancelled to alter the output acoustic signal.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is a diagram of the active noise cancellation unit of the present invention.

FIG. 2 is a perspective rendering of the active noise cancellation unit of the present invention.

Like reference numbers and designations in the various drawings indicate like elements.

### DETAILED DESCRIPTION

It is therefore a teaching of the present invention to recite an active noise cancellation unit which may be mounted or otherwise situated within a plenum or duct fixture of an AHU so as to actively and adaptively cancel the sound energy generated by the components of the AHU. In particular, the active noise cancellation unit of the present invention interposes a speaker or speakers between an input microphone located upstream of the speakers and an error microphone located downstream of the speakers. The active noise cancellation unit of the present invention senses the sound propagated through the system with the input microphone and produces an output audio signal capable of largely canceling the acoustic energy contained within the propagated sound. The output audio signal is broadcast through one or more speakers with the result sensed by the error microphone. The signal received by the error microphone is then used to determine what alteration to the output audio signal is required to most optimally cancel the upstream sound propagation. As a result, the active noise cancellation unit of the present invention works to actively and adaptively cancel sound propagated through the ducts and plenums of AHUs.

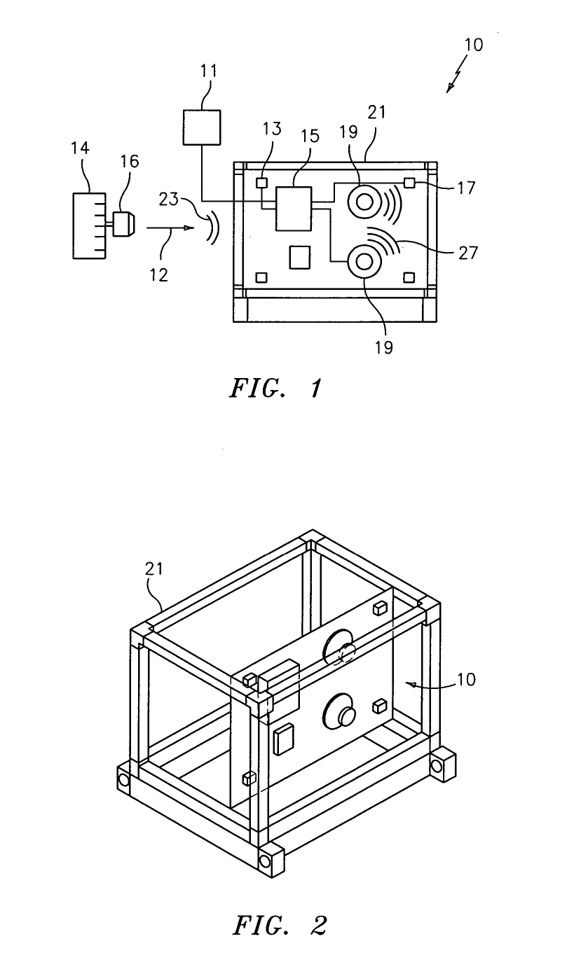

With reference to FIG. 1, there is illustrated an active noise cancellation unit 10 of the present invention. Active noise cancellation unit 10 is shown situated within a plenum 21. While illustrated with reference to a plenum of an AHU, the active noise cancellation unit 10 of the present invention is not so limited. Rather, the active noise cancellation unit 10 of the present invention may be situated and employed in any partially closed space, such as a plenum, through which sound propagates. In the present illustration, airflow 12 moves from left to right through the plenum 21 in which the active noise cancellation unit 10 is situated. Upstream, or against airflow 12, from the active noise cancellation unit 10 is illustrated a fan 14 powered by a motor 16. While the active noise cancellation unit 10 of the present invention is ideally constructed to cancel the noise generated from a motor 16 or fan 14, it is similarly capable of canceling the noise generated from any component capable of producing unwanted acoustic energy or sound.

Sound 23 generated by the fan 14 and motor 16 travels, generally, in the direction of airflow 12 towards the active noise cancellation unit 10. An input microphone 13 operates to convert the sound 23 into an electronic signal. This electronic signal is forwarded as an input to a controller 15. In addition to receiving an input signal from input microphone 13, controller 15 additionally receives input from error microphone 17. Error microphone 17 is located downstream from input microphone 11 and is additionally located downstream from speaker or speakers 19. Error microphone 17 receives a cancelled sound signal resulting from the mixture of the sound 23 and the acoustic output signal 27. In operation, the controller 15 compares the electronic signal received from the input microphone 13 to the electronic signal received from the error microphone 17 and processes this information to compute an output signal for the speakers 19.

Specifically, controller 15 operates to output a signal to the speaker 19 so as to produce an acoustic output signal 27. This acoustic output signal 27 is ideally of a magnitude similar to that of the sound 23 which is to be cancelled, but is 180° out of phase. When sound emitted downstream from a speaker 19 is 180° out of phase with the sound 23 generated by the fan 14 and motor 16 and received by the input microphone 13, the output of the speaker 19 mixes with the sound 23 to be cancelled and results in the cancellation of the sound 23. Because the sound 23 to be cancelled is not constant, but rather may vary due to fluctuations in temperature, pressure, and operation of the fan 14 and motor 16, the output signal sent by the controller 15 to the speaker 19 must be constantly modulated, configured, and adapted to cancel the unwanted sound 23. For this purpose, error microphone 17 is located downstream from the speakers 19 in a region where it is expected that the acoustic output signal 27 from speakers 19 will largely cancel out the unwanted sound 23. If the output sound generated by the speakers 19 perfectly cancels the unwanted sound, error microphone 17 will not detect any sound. However, as the temperature and pressure of the air through which the unwanted sound 23 travels changes, the output signal generated by the speaker 19 will likely move slightly away from a perfect 180° out of phase. This failure to perfectly cancel the unwanted sound can be detected by controller 15 by examining the input from error microphone 17. Controller 15 is adapted to process the residual uncanceled noise detected by error microphone 17 and to adjust the output signal to speaker 19 in such a manner so as to enable speaker 19 to generate an acoustic output signal 27 sufficient to cancel the unwanted noise 23. Under normal operation, the output acoustic signal of speaker 19 closely resembles the sound received by input microphone 13 but is shifted 180° out of phase.

It is not generally sufficient that the output signal generated by the controller 15 and sent to the speaker 19 match the input sound received by input microphone 13 shifted by 180° in phase. This is because speaker 19 is not located at the same point at which input microphone 13 records sound. Rather, there is a period of time required for sound 23 to propagate from input microphone 13 to speakers 19. It is therefore necessary that controller 15 perform a time shift in the signal to be outputted by speaker 19 as well as a phase shift of approximately 180° in order to cancel the unwanted noise.

In a preferred embodiment, controller 15 is a digital signal processor or other microprocessor adapted to receive electronic or digital information from input microphone 11 and error microphone 17 corresponding to digital representations of acoustic signals, and further capable of processing the signals through the application of a transfer function so as to generate an electronic output signal to speakers 19 which may be broadcast as acoustic output signals 27. The output acoustic signals 27 may be of any frequency necessary to attenuate or cancel the sound 23. In a preferred embodiment, the output acoustic signals 27 are approximately between 60 Hz and 300 Hz. More preferably, the frequency of the acoustic signals outputted by speakers 19 are between 63 Hz and 250 Hz.

The active noise cancellation unit 10 of the present invention requires no calibration prior to its installation in a duct or plenum 21 of an AHU. Once activated, the controller 15 works to time shift and phase shift the signal received from input microphone 13 in order to create an output acoustic signal 27 to be broadcast by speakers 19. The calculation of the time and phase shift may be performed using default values for the period of time required for sound to propagate from the input microphone to the speaker 19. To the extent that such a default assumption does not perfectly model the operating environment in which the active noise cancellation unit 10 is operating, the extent of any incongruity may be determined by the controller 15 by analyzing the input received from the error microphone 17 as discussed above. In this manner, the controller 15 may constantly modulate the output signal sent to speaker 19 in order to continually, actively, and adaptively cancel unwanted noise.

In an alternative embodiment of the present invention, a sensor 11 may be utilized to provide additional information to controller 15. Sensor 11 may be directed to recording variables including, but not limited to, temperature and pressure of the airflow 12. Using such information, controller 15 can calculate the speed of propagation of sound through the airflow 12 when calculating the time delay required when producing and emitting an output signal to speaker 19.

With reference to FIG. 2, there is illustrated a perspective rendering of the active noise cancellation unit 10 of the present invention located within a plenum 21.

One or more embodiments of the present invention have been described. Nevertheless, it will be understood that various modifications may be made without departing from the spirit and scope of the invention. Accordingly, other embodiments are within the scope of the following claims.

1. Active noise cancellation apparatus comprising:
a plenum through which a sound travels; and
a noise cancellation unit located within said plenum said noise cancellation unit comprising:
means for receiving said sound;
means for receiving a cancelled sound signal;
means for converting said received sound and said cancelled sound signal into an output signal; and
means for emitting said output signal as an output acoustic signal to cancel said sound.

2. The apparatus of claim 1 wherein both of said output acoustic signal and said sound have a substantially similar magnitude and a substantially similar frequency.

3. The apparatus of claim 1 wherein said output acoustic signal is of a magnitude and frequency sufficient to partially cancel said sound.

4. The apparatus of claim 1 wherein a phase of said output acoustic signal differs from a phase of said sound by approximately 180°.

5. The apparatus of claim 1 wherein said output acoustic signal is between 60 Hz and 300 Hz.

6. The apparatus of claim 5 wherein said output acoustic signal is between 63 Hz and 250 Hz.

7. The apparatus of claim 1 wherein said sound is generated by a component of an air handler unit (AHU).

8. The apparatus of claim 1 wherein said means for receiving said sound comprises a microphone.

9. The apparatus of claim 1 wherein said means for receiving said cancelled sound signal comprises a microphone.

10. The apparatus of claim 1 wherein said means for converting comprises a digital signal processor.

11. The apparatus of claim 1 wherein said means for emitting said output signal comprises a speaker.

12. Active noise cancellation apparatus comprising:
a plenum through which a sound travels; and
a noise cancellation unit located within said plenum said noise cancellation unit comprising:
means for receiving said sound;
means for receiving a cancelled sound signal;
means for emitting an acoustic output signal; and
a controller adapted to convert said received sound and said cancelled sound signal into an output signal to be emitted as an output acoustic signal to cancel said sound.

13. A method for canceling sound in an air handler unit comprising:
receiving said sound;
emitting an output acoustic signal to cancel said sound;
receiving a cancelled sound signal; and
converting said received sound and said cancelled to alter said output acoustic signal.
